(19) United States
(12) Patent Application Publication
(10) Pub. No.: US 2022/0047430 A1
(43) Pub. Date: Feb. 17, 2022

(54) ABSORBENT ARTICLE WITH WETNESS INDICATOR

(71) Applicant: Essity Hygiene and Health Aktiebolag, Göteborg (SE)

(72) Inventor: Claire LUCK, Manchester (GB)

(73) Assignee: Essity Hygiene and Health Aktiebolag, Göteborg (SE)

(21) Appl. No.: 17/413,154

(22) PCT Filed: Dec. 20, 2018
(86) PCT No.: PCT/SE2018/051349

§ 371 Date: Jun. 11, 2021

(51) Int. Cl. A61F 13/42; A61F 13/514; A61F 13/536

(52) U.S. Cl. CPC A61F 13/42; A61F 13/51496; A61F 2013/8497; A61F 2013/422; A61F 13/536

(57) ABSTRACT

An absorbent article including an absorbent core arranged between a topsheet and a backsheet, the absorbent article having a longitudinal axis and a transversal axis extending in a perpendicular direction in relation to the longitudinal axis, the absorbent article having a front portion, a back portion and a crotch portion there between, and at least one wetness indicator, wherein at least one first wetness indicator is arranged in the front portion and/or the back portion. Furthermore, the absorbent article includes at least one permanent visual element arranged in the crotch portion, and the permanent visual element mimics the visual appearance of the wetness indicator.

### TECHNICAL FIELD

The present disclosure relates to an absorbent article, such as a diaper e.g. a pant type diaper or open type diaper for infants or adults or an incontinence article, for male or female users, comprising a wetness indicator.

The present disclosure also relates to a method for manufacturing an absorbent article of the above-mentioned type.

### BACKGROUND

Today, many disposable absorbent articles comprise a wetness indicator, allowing the caretaker to check the wetness state of the disposable absorbent article. Such article is previously known in the art by, for example, US patent application No. US 2010/0262099 A1.

Previously, many disposable absorbent articles positioned the wetness indicator in the crotch portion of the absorbent article. When placed within the crotch portion, the wetness indicator signals the actual wetness of the absorbent article.

However, recent design disclosed in US patent application No. US 2017/0304126A1 for disposable absorbent articles includes a wetness indicator positioned outside of the crotch portion, often referred to as a ‘change indicator’. When placed outside of the crotch portion, the change indicator may be used to signal a need to change the disposable absorbent article for a new one as opposed to conveying knowledge about the actual wetness of the article.

A disadvantage related to positioning of the wetness indicator outside of the crotch portion, is that the caretaker is not aware of its presence. As wetness indicators are traditionally located in the crotch portion of the absorbent article, the caretaker is biased to look for the wetness indicator in the crotch portion. Furthermore, many disposable absorbent articles have a non-functional permanent graphic located on the backsheet. The permanent graphic may for example interact with the wetness indicator as disclosed in the just mentioned application by hiding the wetness indicator until the wetness indicator is wetted. There is however still room for improving the interaction between permanent graphics and a wetness indicator as will be apparent when reading the disclosure.

### SUMMARY

The present disclosure is based on the need to increase the caretaker's awareness of the presence of a change indicator in an absorbent article, and to indicate to the caretaker the location of the change indicator. The present disclosure provides an absorbent article comprising a wetness indicator which before being wetted with body fluids, gives the caretaker the perception of having a greater body fluid uptake area, as compared to the wetness indicator's actual body fluid uptake area.

The disclosure concerns an absorbent article according to claims 1 to 22, a method according to claim 23, and a use according to claim 24. Further embodiments are set out in the dependent claims, in the following description and in the drawings.

At least one of the objectives is achieved by, or at least partly achieved by, an absorbent article comprising an absorbent core arranged between a topsheet and a backsheet. The absorbent article has a longitudinal axis and a transversal axis extending in a perpendicular direction in relation to the longitudinal axis. The absorbent article has a front portion, a back portion and a crotch portion there between. The absorbent article comprises at least one wetness indicator, wherein the at least one wetness indicator is arranged in the front portion and/or the back portion. Furthermore, the absorbent article comprises at least one permanent visual element arranged in the crotch portion mimicking the visual appearance of the wetness indicator. By having a permanent visual element mimicking the visual appearance of the at least one wetness indicator, a user is led to believe that the body fluid uptake area is larger than the actual body fluid uptake area. By the term “body fluid uptake area” as used herein, is meant an area defined by, or in the proximity to, the wetness indicator, at which when subjected to body fluids, the wetness indicator indicates wetness.

The absorbent article may be provided with a permanent visual element to illustrate the presence and position of the wetness indicator to the caretaker. In other words, the absorbent article maybe provided with means to visually communicate to the caretaker the location of the wetness indicator and to make the caretaker aware of the wetness indicator. The permanent visual element may be a set of individual permanent visual elements.

The permanent visual element preferably mimics the visual appearance of the wetness indicator by having at least two of three mimic parameters; color, shape and alignment. It should be noted that any two of these three mimic parameters maybe enough for the permanent visual element to be considered to mimic the visible appearance of the wetness indicator.

The permanent visual element may mimic the visual appearance of the wetness indicator before and/or after use. In an embodiment, the permanent visual element mimics the visual appearance before use only. It is believed without being bound by theory that if the permanent visual element only mimics the visual appearance of the wetness indicator before use, a user is more likely to detect that the wetness indicator is indicating a wet state of the absorbent article.

The wetness indicator may comprise a composition comprising a pH indicator and/or water-soluble dye. It should be noted that if the absorbent article comprises more than one wetness indicator, the wetness indictors may be of the same type or of a different type of wetness indicator. Just as a matter of example, a first wetness indicator may comprise a composition comprising a pH indicator and a second wetness indicator may comprise a composition comprising a water soluble dye.

The wetness indicator may have a shape selected from a polygonal shape, a circular shape or an oblong shape. It should be noted that other shapes are possible also such as animated figures e.g. cartoon figures, animals, buildings, plants such as trees, flowers, or the like. The wetness indicator may be oblong and optionally, generally straight and optionally parallel with the longitudinal axis. A generally straight wetness indicator may facilitate the manufacturing process as it is a simple process requiring little material. A suitable shape of the one or more wetness indicators may be a rectangular shape.

The absorbent article may comprise a graphical element located around at least one wetness indicator wetness indicator to form an inspection window. The wetness indicator inspection window forms an additional visual guidance for the caretaker to locate the wetness indicator.

The wetness indicator may be located between the backsheet and the absorbent core, e.g. by being printed on the backsheet. The wetness indicator may be printed on the body-facing side of the backsheet. The backsheet may be printed in line, i.e. during manufacturing of the absorbent article, or off-line, i.e. introduced to the manufacturing as a pre-printed backsheet.

The wetness indicator may be located only in the front portion or only in the back portion of the absorbent article. Locating the wetness indicator only in the front portion or only in the back portion may be a cost-effective way of providing the absorbent article with a wetness indicator.

The permanent visual element may be a print. The permanent visual element may have a shape selected from a rectangular, a circular or an oblong shape class. It should be noted that other shapes are also possible such as cartoon figures, animals, buildings, plants such as trees, flowers, or the like.

The permanent visual element may be oblong and optionally, generally straight and optionally parallel with the longitudinal axis. By using generally oblong shapes, the permanent visual element may be used to indicate the location of the wetness indicator by use of the direction of the oblong shape. In other words, the oblong visual permanent element may direct the caretaker's attention to the wetness indicator by having the endpoints of the visual permanent element in close proximity to the wetness indicator, or adjacent to the wetness indicator. Optionally, when both the front and the back portion of the absorbent article are provided with an oblong wetness indicator, the permanent visual element may be an oblong shape connecting the endpoints. The permanent visual element may be interconnecting the first and the second wetness indicator. In this embodiment, the permanent visual element mimics the first and the second wetness indicator and provides together with the first and the second wetness indicator the visual appearance of one single wetness indicator. When only one wetness indicator is present, the permanent visual element provides together with the wetness indicator the visual appearance of one single wetness indicator. The wetness indicator area is thus smaller than the area of the wetness indicator(s) and the permanent visual element together, generating a visual appearance of a larger wetness indicator than actually present in the absorbent article.

The permanent visual element may extend generally symmetrically along the longitudinal axis. Optionally or additionally, the permanent visual element may be located on the central longitudinal axis of the absorbent article. In an embodiment, one or more wetness indicators and the permanent visual element may be aligned with the longitudinal axis, or at least parallel with the longitudinal axis. The permanent visual element and the wetness indicator may together extend across 50% or more of the total length of the backsheet of the absorbent article measured along the longitudinal center line, preferably along at least 70%, more preferably along at least 75%, along at least 80%, or along at least 85%.

The permanent visual element and the wetness indicator may be visible through the backsheet. In other words, the permanent visual element and the wetness indicator may be applied on the inside of the backsheet, i.e. the body-facing side, yet be visible by a caretaker facing the garment-facing side of the absorbent article. The permanent visual element may be applied by printing or by being attached to the backsheet e.g. as a sticker.

The permanent visual element and the wetness indicator may have substantially the same alignment. Aligning the permanent visual element with the wetness indicator may further help the caretaker to localize the wetness indicator by highlighting the longitudinal axis on which the wetness indicator is located.

The wetness indicator may have a first width and the centerline of the permanent visual element may be located within a maximum distance of 5 times the first width, such as 3 times the first width, of the longitudinal centerline of the wetness indicator. By having the centerline of the permanent visual element close to the wetness indicator, the visual appearance is that they are connected and relate to each other.

The wetness indicator may have a first width and the permanent visual element has a second width, and the second width of the permanent visual element is between 75-125% of the first width of the wetness indicator, or optionally between 85-115%.

The permanent visual element and the wetness indicator may have the same color, or substantially the same color. The colors of the wetness indicator and the permanent visual element should be selected so that they relate to each other enough for the permanent visual element to mimic the wetness indicator. Just as a matter of example, the color red does not mimic the visual appearance of the color green. However, two different shades of red can be considered to mimic the visual appearance of each other.

According to an embodiment, the color difference ΔE*ab between the wetness indicator in unwetted state and the permanent visual element in unwetted state may be less than 25, such as less than 20. A color difference ΔE*ab of less than 25 ensures that the permanent visual element and the wetness indicator have substantially the same color, or at least be similar enough to be considered to mimic the visual appearance of each other.

The wetness indicator in unwetted state and the permanent visual element may have a difference in a* value of less than 15, and a difference in b* value of less than 15. In other words, both the difference in a* value and the difference in b* value between the wetness indicator in unwetted state and the permanent visual element may be less than 15. This ensures that the permanent visual element and the wetness indicator have substantially the same color.

The wetness indicator and the permanent visual element may extend symmetrically along the longitudinal axis. In other words, the permanent visual element and the wetness indicator may be located on the same longitudinal axis. In this way, the permanent visual element further assists the caretaker in localizing the wetness indicator by highlighting the longitudinal axis on which the wetness indicator is to be found.

The permanent visual element may have substantially the same shape as the wetness indicator. In an embodiment, the permanent visual element is identical to the wetness indicator. As an alternative, the permanent visual element may be similar but not identical to the wetness indicator but still mimicking the visual appearance of the wetness indicator.

The absorbent article may be provided with an absorbent core having one or more absorbent core layers. The absorbent core may be provided with channels, preferably through channels, i.e. extending through the entire thickness of the absorbent core. To reduce the tendency for sagging, the absorbent core may be provided with channel sealings. Just as a matter of example, the channels sealing may be formed by providing a core cover enveloping the absorbent core. The core cover is then secured in the channels e.g. by ultrasonic welding. The absorbent core may be provided with two channels extending along the longitudinal axis. The absorbent core may comprise two channel sealings extending along the longitudinal axis. In other words, the absorbent core of the absorbent article may comprise two channel sealings extending in the same direction as the longitudinal axis. The presence of channel sealings reduces the tendency of the absorbent article to sag in the crotch portion when wetted.

The permanent visual element may be located in between the two channel sealings and/or between a first and a second channel of the absorbent core.

Each channel sealing may have a length which is between 10-75%, such as between 20-60%, such as between 30-50%, of the length of the absorbent core.

The position of the channel sealings along the longitudinal direction of the article is arranged so that the distance between the front edge of the article and the front edge of each channel is between 15-40%, such as 22-25%, of the total length of the article.

The present disclosure also pertains to a method for manufacturing an absorbent article having a longitudinal axis and transversal axis extending in a perpendicular direction in relation to the longitudinal axis and the absorbent article having a front portion, a back portion and a crotch portion. The method for manufacturing comprises the steps of; providing an absorbent core between a topsheet and a backsheet; providing at least one wetness indicator arranged in the front portion and/or the back portion; and providing a permanent visual element in the crotch portion. The manufacturing method further comprises the step of forming the permanent visual element so that it mimics the visual appearance of the wetness indicator.

This disclosure also pertains to the use of a permanent visual element to mimic the appearance of a wetness indicator in an absorbent article.

### BRIEF DESCRIPTION OF THE FIGURES

The present disclosure will be further explained hereinafter by means of examples and with reference to the appended drawings wherein:

FIG. 1 shows a top view of the garment-facing side of an absorbent article in the form of a diaper;

FIG. 2 shows an exploded view of the diaper;

FIG. 3A-3D show several examples of permanent visual elements;

FIG. 4 is a table that illustrates the color differences in ΔE*ab value of different colors.

### DETAILED DESCRIPTION

The term “absorbent article” refers to a product that is configured to be worn on or around the lower torso of the body of a human wearer. The absorbent article is intended to absorb and/or contain body exudates, such as urine, feces and menses fluid. Absorbent articles include for example open absorbent articles, pant-type absorbent articles and belt-type absorbent articles, being able to be worn without the support from underwear. The absorbent article may be a disposable absorbent article.

The “body facing side” of the absorbent article refers to the side facing the wearer during use and the “garment-facing side” refers to the side facing away from the wearer during use. The garment facing side and the body facing side are thus facing away from one another.

An “open type absorbent article” relates to an absorbent article having fastening elements arranged to connect the front region with the back region when the article is being worn.

A “pant type absorbent article” relates to an absorbent article wherein the front region and back region are permanently connected prior to use, forming a waist opening and a pair of leg openings.

By “visual element” it is meant that a subject having a good vision in both eyes (10/10) holding the article at a distance of about 50 cm in a brightly lit room with incandescent light can the see the element. The main function of the visual element is to provide a visual feature to the article.

The term “permanent visual element” as used in this disclosure refers to a visual element that does not substantially change its degree of visibility or visible appearance when insulted with fluids.

The term “mimic” as used in this disclosure refers to how the permanent visual element is made to appear like the wetness indicator. Preferably, the term “mimic” means that the permanent visual element fulfils at least two mimic parameters as defined herein.

A “mimic parameter” refers to a parameter of the permanent visual element that it is substantially the same as when measured on the wetness indicator, or within a given interval. A mimic parameter may refer to color, shape or alignment. The permanent visual element preferably mimics the visual appearance of the wetness indicator by having at least two optionally three mimic parameters, i.e. color, shape and/or alignment.

When referring to color, “substantially the same”, means that the color difference ΔE*ab between the wetness indicator in unwetted state and the permanent visual element is less than 25, such as less than 20. When the difference in the a* values of the colors of the permanent visual element and the unwetted wetness indicator is less than 15, and the difference in the b* values of the colors of the permanent visual and the unwetted wetness indicator is less than 15, their colors are also considered to be substantially the same.

When referring to shape, “substantially the same” may refer to permanent visual elements in which the corresponding angles are equal, e.g. squares and rectangles, or permanent visual elements in which the outline forms the same shape as the wetness indicator. The individual lines forming the outline may be different, e.g. continued lines versus dotted lines or different thicknesses, but when the lines form an outline of the same shape, the permanent visual elements are said to have substantially the same shape. When the outline of the permanent visual element is the same as the outline of the wetness indicator but orientated in a different direction and/or mirrored, the shape of the permanent visual element is also considered to be the substantially the same. Just as a matter of example, a permanent visual element being formed by a figure and a wetness indicator being formed by a similar figure but of different size, the permanent visual element is considered to have the same shape as the wetness indicator. The permanent visual element may of course have the same shape and size as the wetness indicator.

When referring to alignment, “substantially the same” may mean that the permanent visual element extends in the longitudinal direction along the same central longitudinal axis as the wetness indicator.

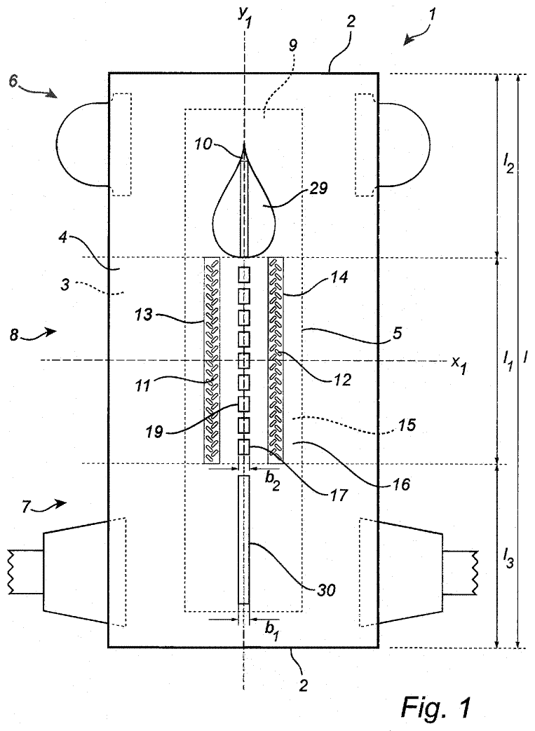

With reference to FIG. 1, permanent visual elements of which the central longitudinal axis y1 is located within a range of 5*b1, such as 3*b1, wherein b1 is the maximum width of the wetness indicator, on each side of the central longitudinal axis y1 of the wetness indicator 29, 30, are also considered to be aligned with the wetness indicator. When the wetness indicator is located in the front portion between the extension of the longitudinal centrelines running through the channels, “substantially the same alignment” may refer to the location of the permanent visual element between the channels. The permanent visual element and the wetness indicator may be arranged so that the central longitudinal axis intersects with the centre of the permanent visual element and the centre of the wetness indicator.

FIG. 1 shows a top view of the garment-facing surface of an absorbent article 1 according to the present disclosure. The absorbent article 1 is an open-type diaper, but the disclosure is not limited thereby. The absorbent article 1 is shown in FIG. 1 in an unfolded and flat state with a view towards a backsheet 4, more specifically the garment facing side of the backsheet 4. The absorbent article 1 has a liquid-permeable topsheet 3, hereafter referred to only as “topsheet”, a liquid-impermeable backsheet 4, hereafter referred to only as “backsheet”, and an absorbent core 5 arranged between the topsheet 3 and the backsheet 4. The topsheet 3 is arranged at the body-facing surface of the absorbent article 1, i.e. at the side facing the wearer, whereas the backsheet 4 is arranged at the garment-facing side of the absorbent article 1. Furthermore, both the topsheet 3 and the backsheet 4 extend laterally outside of the absorbent core 5 along the entire perimeter of the absorbent article 1.

As shown in FIG. 1, the absorbent article 1 has a longitudinal extension along a longitudinal axis y1, also referred to as a central longitudinal axis y1, and a transverse extension along a transverse axis x1, which is perpendicular to the longitudinal axis y1. Furthermore, the absorbent article 1 has a front portion 6, a back portion 7 and a crotch portion 8. The front 6 and back 7 portions each have a waist edge 2. The front portion 6 is intended to be oriented towards the wearer's belly during use of the absorbent article 1 while the back portion 7 is intended to be oriented towards the wearer's back during use. The crotch portion 8 is intended to be positioned between the wearer's legs. The article may furthermore be configured so that one third of the total length of the absorbent article 1 is in the crotch region 8, hence the front portion 6 and the back portion 7 may each be configured to be one third of the total length of the absorbent article 1.

The absorbent core 5 is in the shown embodiment enveloped with a core cover 9. The absorbent core 5 comprises two channels 13, 14 which constitute a section of the absorbent core which are not filled with absorbent material. The two channels 13, 14 are in the shown embodiment thus free of absorbent material. Just as a matter of example, the two channels 13, 14 may be obtained through manufacturing the absorbent core 5 involving a mat forming process during which absorbent material is omitted from the areas which correspond to the two channels 13, 14. In this manner, no absorbent material will be present in the channels 13, 14.

To secure that the void defined by the two channels 13, 14 is maintained during use, the absorbent article 1 may be provided with channel sealings. Two channel sealings 11, 12 are formed in the two channels 13, 14. In FIG. 1, the two channel sealings 11, 12 are illustrated with a fish scale pattern covering the two channels 13, 14 in the absorbent core 5.

The core cover 9 has an upper core cover side 15 and a lower core cover side 16. The channel sealings 11, 12 in FIG. 1 are configured by joining the upper core cover side 15 to the lower core cover side 16 thus creating permanent channels which will not collapse during use. The disclosure is not limited to a core cover comprising two individual core cover layers. The core cover may be of one single material layer. The absorbent core 5 may optionally be enclosed by one core cover layer folded in two, or enclosed by a continuous core cover sheet, thereby providing upper and lower core cover sides for wrapping the absorbent component.

The upper core cover side 15 and the lower core cover side 16 may be attached to each other by various technologies, for example, by thermo bonding, mechanical bonding, thermo-sealing, ultrasonic bonding, adhesive, stitching, or the like, or a combination thereof. With reference to the embodiment shown in FIG. 1, the upper core cover side 15 and the lower core cover side 16 are attached to each other by ultrasonic welding.

With reference again to FIG. 1, it can be noted that two channel sealings 11, 12 may have a length 11 which corresponds to the longitudinal extension of the crotch portion 8. Hence in an embodiment, the absorbent core may be provided with one or more channels, preferably two or more channels, in which the channels have a length corresponding to the length of the crotch portion of the absorbent article. Furthermore, the channel sealings 11, 12 may have a length 11 which is between 10-75%, such as between 20-60%, such as between 30-50%, of the length of the absorbent core 5.

The positioning of the channels 13, 14 and/or the channel sealing 11, 12 may be at a first distance l2 from the waist edge 2 at the front portion 6 and at a second distance 13 from the waist edge 2 of the back portion 7. The channel sealings 11, 12 may be positioned at different positions along the longitudinal axis of the absorbent article 1. Such positioning may be defined by choosing a suitable value for the distance l2 between the waist edge 2 of the front portion 6 and each channel sealing 11, 12. Obviously, this means that decreasing said distance l2 means that a distance l3 between the waist edge 2 of the back portion 7 and the channel sealings 11, 12 will be increased, and vice versa.

The position of the channel sealings 11, 12 along the longitudinal direction of the absorbent article 1 may be chosen in a manner so that the distance l2 between the waist edge 2 of the article 1 and each channel 11, 12 is between 15-40%, optionally between 30-40%, such as between 22-25%, of the total length l of the article 1.

The absorbent article 1 in FIG. 1 includes an absorbent core 5 having a rectangular design. However, the absorbent core is not limited to this form but may be formed in generally any geometric form.

The absorbent article 1 is provided with one or more wetness indicators. In FIG. 1, the absorbent core 5 comprises a first wetness indicator 29 present only in the front portion 6 and a second wetness indicator 30 arranged only in the back portion 7. The absorbent article is thus arranged with two distinct wetness indicators. Alternatively, the absorbent core 5 may comprise only one wetness indicator present only in the front portion 6 or only in the back portion 7. In other words, the absorbent core 5 comprises a wetness indicator 29 positioned only in the front portion 6, or a wetness indicator 30 positioned only in the back portion 7, or both these wetness indicators 29, 30 positioned only in front portion 6 and back portion 7, respectively.

In the following, the reference will be made primarily to the wetness indicator as indicated by reference number 29 in the drawings. However, the disclosure applies in the same manner to any other wetness indicator as disclosed herein such as the second wetness indicator 30 arranged in the back portion 7 of the absorbent article 1.

The one or more wetness indicators may be constituted by a relatively thin layer of a composition, such as an adhesive composition, comprising a colorant such as a pH-indicator or a water-soluble dye. One such wetness indicator is Kmelt W 2488 OL from Colquimica S.A. The wetness indicator may be sensitive to moisture and/or pH changes and changes appearance/color when it is wet. In this manner, the wetness indicator 29, 30 may be configured for providing a visual signal when in contact with urine. The wetness indicator may be applied by slot coating.

FIG. 1 shows an absorbent article 1 having a wetness indicator 29 in the shape of a rectangle. However, the wetness indicator 29 may have any geometrical shape e.g. triangle, square, rectangle, diamond, other polygonal shapes, circle, elliptic, oval, heart, star, or a combination thereof. Alternatively, the wetness indicator may be a non-geometrical figure such as an animal, a boat, a car, an animated figure or the like. In the shown embodiment the wetness indicator 29 is not arranged between the two channels 13, 14. The absorbent article 1 is thus provided with a wetness indicator free area between first and a second channel of the absorbent core.

The absorbent article 1 comprises a permanent visual element. As illustrated in FIG. 1, the crotch region 8 comprises a set of permanent visual elements 19 formed by a plurality of rectangular graphical elements 17. The number of graphical rectangular elements 17 may vary from 2-40, preferably from 5-20 graphical rectangular elements. As can be noted in FIG. 1, the plurality of graphical rectangular elements 17 are aligned with the longitudinal axis y1, just as the wetness indicator 29. In this sense the graphical rectangular elements 17, and thus the permanent visual element 17, appears to be an extrapolated part of the wetness indicator 29 extending across the crotch region 6 of the absorbent article 1. In practice the set of permanent visual elements 19 has however no wetness indicating function but merely operate to mimic the visual appearance of a wetness indicator in the crotch region 5, and thus the presence of a wetness indicator in the crotch region 5. The graphical rectangular elements 17 together forms, as can be gleaned from FIG. 1, the substantially the same shape as the wetness indicator 29.

The permanent visual element 17 may be a colour print. The colour print may for example be realized by means of analogue or digital printing technology. Analog printing technology here refers to the use of printing plates or rolls that are coated with colour liquid and subsequently pressed on a surface to be printed, such that the surface to be printed is provided with a colour printing pattern. Digital printing technology typically refers to inkjet printing where ink is transferred onto the substrate to create the desired image, electrophotography printing where toner is attracted to a charged image and then developed, or laser printing. Where digital printing is used the colour printing pattern may be accomplished by printing a large number of small colour dots very close to each other. The resolution of the colour printing pattern may be defined in terms of dots per inch (DPI). A disc with a diameter of 1.5 millimeters mapped onto a printed surface area is not deemed extending beyond a periphery of the printed surface area merely because the printed surface area has a low resolution, when the printed surface area extends both in the longitudinal direction x and transverse direction and when the resolution is at least 50 DPI, specifically at least 100 DPI, both in the longitudinal and transverse direction x, y. The permanent visual element 17 may have any geometrical shape e.g. triangle, square, rectangle, diamond, other polygonal shapes, circle, elliptic, oval, heart, star, or a combination thereof. Alternatively, the wetness indicator may be a non-geometrical figure such as an animal, a boat, a car, an animated figure or the like.

The permanent visual element mimics the appearance of the one or more wetness indicator(s). By mimicking the appearance of the one or more wetness indicators, the permanent visual element makes the user aware of the presence and location of the wetness indicator.

The permanent visual element 17 in FIG. 1 preferably mimics the visual appearance of the wetness indicators 29 and 30 by having at least two of three mimic parameters, i.e. color, shape and/or alignment. By mimicking the wetness indicator 29 and 30 with at least two mimic parameters, the permanent visual element 17 provides the caretaker with a strong indication of the location of the wetness indicator. However, the permanent visual element 17 may also mimic the wetness indicator by having at least 3 mimic parameters.

In FIG. 1, the permanent visual element 17 is printed on the garment-facing side of the backsheet 4. However, the permanent visual element 17 may optionally be printed on the body-facing side of the backsheet 4.

The permanent visual element 17 in FIG. 1 is located between the two channel sealings 11, 12. When located between the channel sealings, the permanent visual element may indicate the location of the wetness indicator in a more precise manner. Furthermore, there is no need for arranging a wetness indicator between the two channels as little is gained from a care takers perspective as the wetness indicators in the back and/or front portion of the absorbent article are fully operational to indicate the presence of body fluid in the absorbent core of the absorbent article.

The colour of the unwetted permanent visual element 17 in FIG. 1 is substantially the same as the colour of the unwetted wetness indicator 29.

Furthermore, the permanent visual element 17 has substantially the same alignment as the wetness indicator 29 and 30 as they are centered around the same longitudinal axis y1. Moreover, the width b2 of the permanent visual element 17 is between 90-110% of the width b1 of the wetness indicator 29.

The shape of the permanent visual element 17 in FIG. 1 is substantially the same as the shape of the wetness indicators 29 and 30. The outline of the shape of the permanent visual element 17 is the same as the outline of the wetness indicators 29 and 30 as they are all rectangles.

It should be noted that a permanent visual element consisting of several individual permanent visual elements may also mimic the shape of the wetness indicator when the individual permanent visual elements cooperate to form a certain shape, e.g. several dots may cooperate to form the shape of a rectangle, as is the case in FIG. 1 for example.

The absorbent article 1 shown in FIG. 1 furthermore has a wetness indicator inspection window 10 arranged in the front portion 6 of the absorbent article 1. As used herein, the term “wetness indicator inspection window” refers to a graphical permanent element located around the wetness indicator. By “located around” it is meant that part of the graphical element is present in each direction relative to the wetness indicator and thus surrounds the wetness indicator. The wetness indicator inspection window 10 in FIG. 1 is in the form of a droplet. However, the wetness indicator inspection window 10 may have any geometrical shape, e.g. triangle, square, rectangle, diamond, other polygonal shapes, circle, elliptic, oval, heart, star. The wetness indicator inspection window 10 shown in FIG. 1 is in the form of a droplet, which further aids the caretaker in localizing the wetness indicator 29 by appealing to his ability to make a mental connection between droplets and wetness. Although the wetness indicator inspection window 10 may also mimic the visual appearance of the wetness indicator, it should be understood that the wetness indictor inspection window 10 may differ from the permanent visual element by being present only in the front or the back portion. Disclosed herein is also an absorbent article comprising a topsheet, a backsheet and an absorbent core arranged between the topsheet and the backsheet. The backsheet comprising a wetness indicator arranged in a wetness indicator inspection window formed by a graphical permanent element. The graphical permanent element having the shape of a droplet to indicate the function of the wetness indicator arranged within the wetness indicator inspection window.

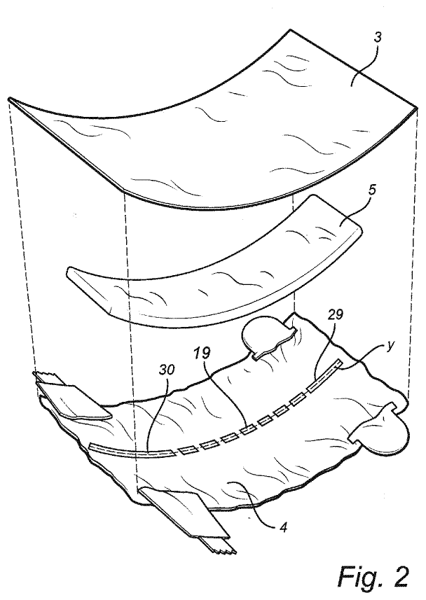

FIG. 2 shows the absorbent article 1 of FIG. 1 in an exploded view showing, from top to bottom; the topsheet 3, the absorbent core 5, and the backsheet 4. The wetness indicator 29 may be arranged in the absorbent core 5, but alternatively, may be located between the backsheet 4 and the absorbent core 5 or on the backsheet 4.

Various types of materials may be used for the absorbent article 1. The topsheet 3 may be formed by a fluid permeable nonwoven fabric or film, which may be made of thermoplastic synthetic fibers. The topsheet 3 may be sufficiently liquid-permeable to allow discharged body fluids to penetrate through the thickness of the topsheet 3. Also, the topsheet 3 may be suitably manufactured from a material which is compliant and soft-feeling to the skin of the wearer. The topsheet 3 may consist of a single layer or have a laminate structure comprising a plurality of layers, for example, two or more layers. The layers may be made of the same material, or some or all the layers may be made of different materials.

The topsheet 3 may be made of a single material or have plural portions made of different materials, e.g. within different parts of the body-facing side of the topsheet 3. The topsheet 3 may be, for example, a hydrophilic, non-apertured nonwoven web of fibers, such as natural fibers, e.g., cotton or pulp fibers, synthetic fibers, e.g., polyester or polypropylene fibers, or a combination of these fibers. The topsheet 3 may have a basis weight in the range of 8-40 g/m 2.

The backsheet 4 may be formed by a liquid-impermeable and breathable layer such as a polymeric film, for example a film of polyethylene or polypropylene. According to different embodiments, the materials which may be used for the backsheet 4 include thin and flexible fluid impermeable plastic films, or fluid impermeable nonwoven materials, fluid impermeable foams and fluid impermeable laminates.

The backsheet 4 may be formed by a single layer, but may alternatively be formed by a laminate, wherein at least one layer is fluid impermeable. Furthermore, the backsheet 4 may be elastic in any direction. Furthermore, the backsheet 4 may have a laminate structure comprising a liquid barrier sheet and a nonwoven layer arranged on top of each other (not shown in detail in the drawings), wherein the nonwoven layer is arranged at an outer side away from the wearer of the absorbent article when worn.

The nonwoven layer of the backsheet 4 in case such is present may be made of thermoplastic polymer material fibers or filaments. The nonwoven layer may be formed by a variety of different processes, such as spunbonding, airlaying, meltblowing or bonded carded web formation processes. The nonwoven layer may be made of an SMS (spunbond/meltblown/spunbond) or SS (spunbond/spunbond) nonwoven material of polypropylene or bicomponent fibers of polypropylene and polyethylene, or of a combination of such materials.

The liquid barrier sheet may be made of a plastic material, for example a thermoplastic film material, and/or a nonwoven material. For example, the liquid barrier sheet may be formed as a plastic layer, e.g., a thermoplastic layer, or a plastic film, e.g., a thermoplastic film. Forming the liquid barrier sheet of a plastic material, such as a thermoplastic film material, allows for a particularly good printability of the liquid barrier sheet. The liquid barrier sheet may also contain paper fibers.

The liquid barrier sheet may be a liquid impermeable, breathable or non-breathable layer. The liquid barrier sheet may consist of a single layer or have a laminate structure with a plurality of layers, e.g., two or more layers, three or more layers, or four or more layers. The layers of the liquid barrier sheet may be laminated, bonded or attached to each other, for example, by thermo and/or mechanical bonding, such as thermo-sealing, ultrasonic bonding, such as ultrasonic welding, an adhesive or adhesives, stitching or the like.

The liquid barrier sheet may be a breathable microporous film. The microporous film may be made of a material comprising at least two basic components, namely a thermoplastic elastomeric polyolefin polymer and a filler. These components and, in some embodiments, additional other components may be mixed together, heated and subsequently extruded into a mono-layer or multi-layer film using any one of various film-producing processes, such as cast embossed, chill and flat cast, and blown film processes.

The absorbent core 5 may be made of one layer only, made from any suitable absorbent or liquid uptake material, such as one or more layers of cellulose fluff pulp, foam, fiber waddings or the like.

The absorbent core 5 may comprise suitable amounts of superabsorbent particles. Superabsorbent material is constituted by a water-swellable and water-insoluble material which is capable of absorbing large quantities of fluid upon formation of a hydrogel. The absorbent core 5 may contain superabsorbent material in the form of fibers or particles of absorbent polymer material. For example, the superabsorbent material may be surface cross-linked, partially neutralized polyacrylates. In an embodiment, the absorbent core comprises one or more layers of superabsorbents. The absorbent core may for example be formed by a single layer comprising 100% weight superabsorbents. Combinations of cellulose fluff absorbent layer and one or more layers of superabsorbent is of course feasible. Furthermore, a core cover may surround the core and may be made of nonwoven material, with a basis weight of 5-20 g/m2.

The superabsorbent material, e.g., the superabsorbent fibers or particles, may be mixed with other absorbent or liquid uptake material or materials, such as cellulose fluff pulp, and/or arranged in pockets or layers in the absorbent core 5. Just as a matter of example, the absorbent core may comprise 10% weight or more, such as 20% weight or more, 40% weight or more, 60% weight or more, or 80% weight or more of superabsorbents, remaining parts being cellulose fluff pulp or any other material as mentioned above

The absorbent core 5 may further comprise components for improving the properties of the absorbent core. For example, the absorbent core 5 may comprise a binder or binders, such as binder fibers. Furthermore, the various layers of the absorbent article 1 may be attached by means of adhesive material.

One or more additional layers may be provided in the absorbent article. For example, an acquisition layer may be arranged between the absorbent core 5 and the topsheet 3. Such an additional layer may for example be in the form of an airlaid layer, a spunlace layer, a high-loft, foam or any other type of material layer which may be used in an absorbent article to act as a liquid acquisition and absorption layer. The acquisition layer is adapted to quickly receive and temporarily store discharged liquid before it is absorbed by the absorbent core 5. Such acquisition layer may be composed of for example airlaid nonwoven, spunlace nonwoven, high loft nonwoven or foam materials. An airlaid nonwoven may be produced with fluff, wood pulp, and here the fluff fibres are dispersed into a fast-moving air stream and condensed onto a moving screen by means of pressure and vacuum.

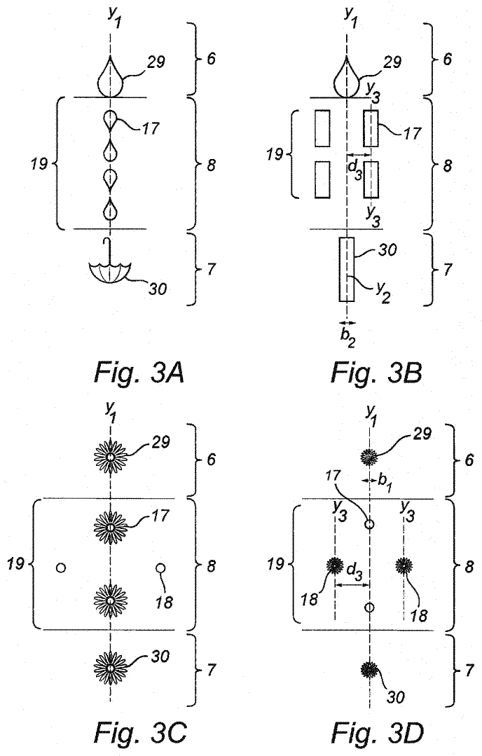

FIGS. 3A-3D shows embodiments of how the permanent visual element mimics the wetness indicator of the absorbent article. For the purpose of clarity, FIGS. 3A-3D only show the permanent visual element and the wetness indicator(s). FIG. 3A shows a first wetness indicator 29 located in the front portion 6 of the absorbent article and a second wetness indicator 30 located in the back portion of the absorbent article. The first wetness indicator 29 is in the form of a droplet, whereas the second wetness indicator 30 is in the form of an umbrella. The set of permanent visual elements 19 is located in the crotch portion and consists of several permanent visual elements 17 orientated along the longitudinal axis y1. Every second permanent visual element 17 changes orientation by 180 degrees. In FIG. 3A, the permanent visual element 17 mimics the wetness indicator 29 by fulfilling two mimic parameters, i.e. shape and alignment. As shown in FIG. 3A, the shape of the permanent visual element 17 is substantially the same as the shape of the wetness indicator 29. Although the orientation of the permanent visual element 17 is different, the outline of the shape is the same as the outline of the shape of the wetness indicator 29. The wetness indicator 29 and the permanent visual element 17 are substantially aligned as their respective centres are located on the longitudinal axis y1. In FIG. 3A, the colours of the wetness indicator 29 and the permanent visual element 17 differ. The visual permanent element 17 has only 1 mimic parameter with respect to the wetness indicator 30, i.e. alignment. However, the visual permanent element 17 aids in the localization of the wetness indicator 30 appealing to the caretakers' mental ability to make a connection between the shape of the permanent visual element 17 in the form of a droplet, and the shape of the wetness indicator 30 in the form of and umbrella. Since the permanent visual element has only 1 mimic parameter with respect to the wetness indicator 30, it is not considered to mimic the visual appearance of the wetness indicator 30 in the back portion.

FIG. 3B shows a first wetness indicator 29 in the form of a droplet located in the front portion 6 of the absorbent article, a second wetness indicator 30 in the form of a rectangle in the back portion 7 and a set of permanent visual elements 19 located in the crotch portion. The permanent visual element 17 mimics the wetness indicator 30 present in the back portion 7 by fulfilling two mimic parameters: shape and alignment. The permanent visual element 17 has substantially the same shape as the wetness indicator 30 because both the permanent visual element 17 and the wetness indicator 30 have a rectangular outline. The wetness indicator 30 located in the back portion has a width b2 and a longitudinal centreline y2. The centreline y3 of the permanent visual element 17 is located at a distance d3 from the longitudinal axis y2 of the wetness indicator 30. The distance d3 is less than 5* b2, thus the permanent visual element 17 has substantially the same alignment as the wetness indicator. Since the permanent visual element 17 has two mimic parameters with respect to wetness indicator 30, it is considered to mimic the appearance of the wetness indicator 30. In FIG. 3B, the colour of the permanent visual element 17 and the wetness indicator 30 differs. It should be noted that the permanent visual element 17 does not have any mimic parameters with respect to the wetness indicator 29 present in the front portion. However, the technical effect of providing a sense of security to the caretaker may be accomplished when the permanent visual element mimics only one of the two wetness indicators present in the absorbent article.

In FIG. 3C, the set of permanent visual elements 19 consists of several individual permanent visual elements 17 and 18 of different shape and colour. The individual permanent visual element 17 mimics the wetness indicator 29 and 30 by fulfilling two mimic parameters: shape and alignment. The individual permanent visual element 18 has only 1 mimic parameter, i.e. colour and therefore does not mimic the wetness indicator. However, the set of permanent visual elements 19 may mimic the wetness indicator by having only 1 individual permanent visual element 17 with 2 mimic parameters. In FIG. 3C, the permanent visual element 17 has the same outline as the wetness indicator and is centred along the same longitudinal axis y1 as the wetness indicators 29 and 30. The wetness indicator 29 located in the front portion and the wetness indicator 30 located in the back portion have the same shape, colour and alignment. Therefore, the permanent visual element 17 mimics both wetness indicators 29 and 30. By mimicking both wetness indicators, the permanent visual element provides a strong level of awareness to the caretaker about the presence of wetness indicator in the absorbent article.

In FIG. 3D, the set of permanent visual elements 19 consists of several individual permanent visual elements 17 and 18 in different shapes and colors. The individual permanent visual element 18 mimics the wetness indicator by fulfilling 2 mimic parameters, i.e. color and shape. As the distance d3 between the longitudinal centerline y3 of the individual permanent visual element 18 is greater than 5*b1, the permanent visual element does not fulfil the mimic parameter “alignment”. The individual permanent visual element 17 in FIG. 3D has only 1 mimic parameter, i.e. alignment, and therefore does not mimic the wetness indicator 29 or 30. The combination of one mimic parameter associated with one individual permanent visual element with another mimic parameter of a different individual permanent visual element to reach a number of two mimic parameters does not achieve the technical effect of mimicking the wetness indicator. Thus, combining the mimic parameter “alignment” of the individual permanent visual element 17 in FIG. 3D with the mimic parameter “color” of the individual permanent visual element 18 in FIG. 3D does not make the set of permanent visual elements 19 mimic the wetness indicator. However, since the permanent visual element 18 has two mimic parameters, the permanent visual element 18 mimics the wetness indicator. As disclosed earlier in this teaching, the set of permanent visual elements 19 may mimic the wetness indicator when only 1 of several individual permanent visual elements mimics the wetness indicator.

A method for manufacturing an absorbent article 1 will now be described. The method comprises the steps of:
Arranging the absorbent core 5 between topsheet 3 and backsheet 4;
Providing at least one wetness indicator 29 in the front portion 6 and/or back portion 7; and
providing a permanent visual element 17 in the crotch portion 8.

The permanent visual element 17 is formed so that it mimics the visual appearance of the wetness indicator 29.

The disclosure also relates to the use of a permanent visual element so that it mimics the appearance of a wetness indicator in an absorbent article.

Measurements

Colour may be determined within the L*a*b* colour space, as established by the Commission Internationale de l'Eclairage (CIE) in 1976. The colour space is divided into three axes. L* represents lightness and the axis extends from 0 (black) to 100 (white). The axis a* goes from green to red, where positive values indicate more saturated red, and negative values more saturated green. The b*-axis goes from blue to yellow, where positive values represent more saturated yellow and negative values more saturated blue. This colour space is well known in industry and is generally referred to as CIE L*a*b* or CIELAB (1976).

A suitable spectrophotometer is available from Konica-Minolta, under the designation CM-5 (equivalent apparatuses can also be used). The apparatus illuminates the sample diffusely and detects the light at 8 degrees to the normal line (a geometry named di:8°, de:8°). The instrument is set to SCE (specular component excluded). Standard Illuminant D65 is utilized, and the viewing angle is set to 10°. Normally the diameter of the measured area should be 30 mm. However, if the relevant sample areas are smaller (having a diameter less than 30 mm) then smaller spectrophotometer apertures are utilized (as large as possible without transgressing the relevant colour area). The tested object is placed flat and smooth against the spectrophotometer aperture. A white ceramic tile (having L* a*b* values at or close to L* 96.1, a* 0.1, b* 2.85) is placed behind the tested object, as a standard backing. Such ceramic tiles are available e.g. from Konica-Minolta.

A difference between two colours in the colour space CIE L*a*b* is characterised by a Delta E-value (ΔE*ab). The differences between the colours on the three respective axes are squared in this, following which the differences are summed and the root derived from the sum:

ΔE_{ab}*=(L₂*−L₁*)²+(a₂*−a₁*)²+(b₂*−b₁*)²

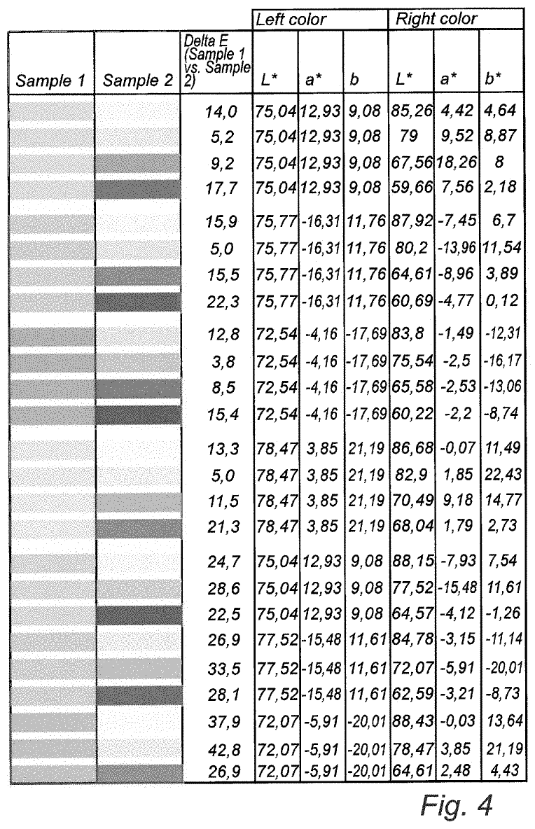

The table in FIG. 4 illustrates the color differences in ΔE*ab value of different colors calculated according to the aforementioned formula. The colours in the FIG. 4 are to be considered as illustrative of the actual colours defined by the values in the table. The colours in FIG. 4 should only be used to better understand the mimic parameter of colour and the internal relationship between different colours. The colour stripes were printed on white standard office paper (80 g/m²), and a conventional spunbond nonwoven (16 g/m²) was placed on top. This configuration simulates a diaper backsheet laminate, where a nonwoven layer covers a film comprising colored indicators and graphical elements.

### Example

The following procedure is applied when measuring the colours on a diaper. Remove the diaper from an unopened package and perform the colour measurements within three hours from opening of the package. The indicator should not be pre-exposed to water or moisture in any way. The laboratory temperature should be about 23° C., and the relative humidity must not exceed 50%. Cut away the chassis and peripheral materials from the diaper and separate the backsheet from the absorbent core. Take care not to disturb the indicator or the relevant colored areas. If the indicator or colored areas are coalesced with parts of the core, e.g. fibers), then let these parts of the core remain stuck to the backsheet. Place the outside of the backsheet (the side seen by someone changing the diaper) with the relevant colored area against the spectrophotometer aperture, and then place the standard white ceramic tile behind the sample. Then measure according to the apparatus instruction.

The open-type baby diaper is of a similar construction as the baby diaper described in connection with FIGS. 1-2. The topsheet is liquid-permeable and is of a spunbonded, 16 gsm hydrophilic material. For this open-type diaper, a spunbond non-woven 16 gsm from Fitesa was used. The backsheet is a laminate of two nonwoven layers formed from hydrophobic spunbond and having a weight per unit area of 18 gsm (Gram per Square Meter). The open-type baby diaper has a white absorbent core surrounded by a white core cover. The wetness indicator is the water-soluble dye Kmelt W2488 OL from Colquimica S.A applied in the form of a yellow line. The permanent visual element is an inkjet print of yellow rectangles forming a dotted line. The colour measurement of the permanent visual element is taken in the centre of one of the rectangles constituting the permanent visual element. The following values were obtained for the permanent visual element: L*75.22; a*2.38 and b*22.9. The following values were obtained for the wetness indicator: L*79.3; a*-5.47; b*33.98. The ΔE*ab value for the color difference between the permanent visual element and the wetness indicator is calculated using the formula described earlier, yielding a value of 14.2.

The absorbent article 1 may additionally be provided with further components such as fastening tabs, standing gathers, elastic elements and other components which are used in absorbent articles such as for example baby diapers or incontinence garments. Such additional components are not described in detail here.

1. An absorbent article comprising an absorbent core arranged between a topsheet and a backsheet, said absorbent article having a longitudinal axis and a transversal axis extending in a perpendicular direction in relation to said longitudinal axis, said absorbent article having a front portion, a back portion and a crotch portion there between, and at least one wetness indicator, wherein said at least one wetness indicator is arranged in said front portion and/or said back portion, and wherein at least one permanent visual element is arranged in said crotch portion,
wherein said permanent visual element mimics the visual appearance of said wetness indicator,
wherein said absorbent core comprises at least two channels, optionally said absorbent core comprises two channel sealings extending along the longitudinal axis.

2. (canceled)

3. (canceled)

4. (canceled)

5. The absorbent article according to claim 1, wherein said wetness indicator is located between the backsheet and the absorbent core.

6. The absorbent article according to claim 1, wherein said wetness indicator is located only in the front portion or back portion.

7. The absorbent article according to claim 1, wherein said permanent visual element is a print.

8. (canceled)

9. The absorbent article according to claim 1, wherein said permanent visual element is oblong and optionally, generally straight and optionally parallel with the longitudinal axis.

10. (canceled)

11. The absorbent article according to claim 1, wherein said permanent visual element and said wetness indicator are visible through the backsheet.

12. The absorbent article according to claim 1, wherein said permanent visual element and said wetness indicator are substantially aligned.

13. The absorbent article according to claim 1, wherein said wetness indicator has a first width and wherein a centerline of the permanent visual element is located within a maximum distance of 5 times said first width, optionally within a distance of 3 times said first width, of said longitudinal centerline of said wetness indicator.

14. The absorbent article according to claim 1, wherein said wetness indicator has a first width and the permanent visual element has a second width, and the second width is between 75-125% of said first width.

15. The absorbent article according to claim 1, wherein said wetness indicator in unwetted state and said permanent visual element have a color difference ΔE*ab of less than 25.

16. (canceled)

17. The absorbent article according to claim 1, wherein said wetness indicator and said permanent visual element extend symmetrically along said longitudinal axis.

18. The absorbent article according to claim 1, wherein said permanent visual element has substantially the same shape as said wetness indicator.

19. (canceled)

20. The absorbent article according to claim 1, wherein said permanent visual element is located in between said two channels.

21. The absorbent article according to claim 1, wherein each channel or optionally channel sealing, has a length which is between 10-75% of the length of the absorbent core.

22. The absorbent article according to claim 1, wherein the position of said channels, or optionally channel sealings, along the longitudinal direction of the article is arranged so that the distance between the waist edge of the article and each said channels, or optionally channel sealing, is between 15%-40% of the total length of the article.

23. (canceled)

24. (canceled)

25. The absorbent article according to claim 1, wherein the permanent visual element mimics the visual appearance before use.
